(19) United States
(12) Patent Application Publication
(10) Pub. No.: US 2014/0275782 A1
(43) Pub. Date: Sep. 18, 2014

(54) ACTIVATION OF IMAGING CAPSULES WITH ALTERNATING CURRENT

(71) Applicant: CHECK CAP LTD., Isfyia (IL)

(72) Inventor: Yoav KIMCHY, Haifa (IL)

(73) Assignee: CHECK CAP LTD., Isfyia (IL)

(21) Appl. No.: 14/218,961

(22) Filed: Mar. 18, 2014

Related U.S. Application Data
(60) Provisional application No. 61/802,377, filed on Mar. 15, 2013.

(51) Int. Cl. A61B 1/00; A61B 1/04

(52) U.S. Cl. CPC A61B 1/00029; A61B 1/041
USPC 600/109

(57) ABSTRACT

An imaging capsule activating system includes a capsule containing a radiation source and an arrangement with the radiation source mounted thereon. The arrangement is configured to selectively allow emission of radiation from the radiation source. A blocking arrangement is configured to selectively block emission of radian from the radiation source. In a rest position, the emission of radiation from the arrangement is blocked. A movement arrangement configured to move the blocking arrangement relative to the arrangement. A controller operates the radiation source and the blocking arrangement. An activation coil is electrically coupled with the controller and has an initial configuration without current flow. An external activating coil receives the imaging capsule and is operable to inductively induce current flow in the activation coil within the capsule.

### CROSS-REFERENCE TO RELATED APPLICATIONS

This application claims the benefit of U.S. Provisional Patent Application No. 61/802,377, having a filing date of 15 Mar. 2013, which is incorporated herein by reference.

### TECHNICAL FIELD

The present disclosure relates generally to imaging capsules and, more specifically, to activation of imaging capsules with alternating current.

### BACKGROUND

One method for examining the gastrointestinal tract for the existence of polyps and other clinically relevant features that may indicate regarding the potential of cancer is performed by swallowing an imaging capsule that will travel through the tract and view the patient's situation. In a typical case the trip can take between 24-48 hours, after which the imaging capsule exits in the patient's feces. Typically the patient swallows a contrast agent to enhance the imaging ability of the imaging capsule. Then the patient swallows the imaging capsule to examine the gastrointestinal tract while flowing through the contrast agent. The imaging capsule typically includes a radiation source, for example including a radioisotope that emits X-rays or Gamma rays. The radiation is typically collimated to allow it to be controllably directed toward a specific area during the imaging process. In an exemplary case the imaging capsule is designed to measure Compton back-scattering and transmits the measurements (e.g. count rate) to an external analysis device, for example a computer or other dedicated instruments.

In a typical implementation a radio-opaque contrast agent is used so that a position with a polyp will have less contrast agent and will measure a larger back-scattering count. Alternatively, other methods may be used to image the gastrointestinal tract.

U.S. patent application Ser. No. 7,787,926 to Kimchy, the disclosure of which is incorporated herein by reference, describes details related to the manufacture and use of such an imaging capsule.

Use of an imaging capsule requires a power source to be connected to the capsule electronics to supply power to the electronics for operation. If the capsule is stored prior to use with the capsule electronics in an active condition, the electronics may draw power from the batteries and/or experience current leakage. If the power source were to fail, the imaging capsule can malfunction in various ways. For example, if the capsule includes an actuatable shutter for blocking radiation when such radiation is not desired, radiation may be emitted without constraint causing potential harm to the patient.

If the imaging capsule is equipped with an internal switch for activating the capsule electronics, such activation may require access to an interior of the capsule. Such a breach of the capsule could leave the capsule in an unsealed, vulnerable condition for use by a patient.

It may thus be desirable to provide an activation apparatus and method that allow the capsule electronics to be connected to the batteries and remain with no power and no current leakage in a sealed shell while the capsule is in storage and enable activation of the electronics without breaching the capsule shell, thus leaving it sealed.

### SUMMARY OF THE INVENTION

This disclosure concerns the description of activation mechanisms for activating the electronic circuits of an imaging capsule. The capsule is designed to be swallowed by the patient and travels through the Gastro Intestinal tract. An example of such a concealment mechanism is described in U.S. patent application Ser. No. 10/596,065, filed on May 26, 2006, now U.S. Pat. No. 7,787,926, titled Intra Lumen Imaging Capsule, and PCT Publication No. WO 2012/035528, titled Fail-safe Radiation Concealment Mechanism, the disclosures of which are incorporated herein by reference.

The concealment mechanism is designed with shutters that are normally closed, effectively stopping the emitted radiation from the radiation source within the capsule to exit the capsule, thus reducing the exposure of the patient to ionizing radiation.

When the radiation is emitted and the collimator is moving and scanning, detectors (13) in FIGS. 1-4 detect X-ray Fluorescence and Compton scattering photons which are used for 3D imaging within the colon as described in U.S. Pat. No. 7,787,926.

The described mechanisms open the shutters only when the capsule requires these photons (or beta electrons) for imaging the internal lumen of the gastro intestinal tract.

According to various aspects of the disclosure, an imaging capsule activating system includes a capsule containing a radiation source, and an arrangement with the radiation source mounted thereon. The arrangement is configured to selectively allow emission of radiation from the radiation source. A blocking arrangement is configured to selectively block emission of radian from the radiation source. In a rest position, the emission of radiation from the arrangement is blocked. A movement arrangement configured to move the blocking arrangement relative to the arrangement. A controller operates the radiation source and the blocking arrangement. An activation coil is electrically coupled with the controller and has an initial configuration without current flow. An external activating coil receives the imaging capsule and is operable to inductively induce current flow in the activation coil within the capsule.

In accordance with the disclosure, a method of activating an imaging capsule includes providing a capsule similar to that described above, disposing the imaging capsule in an open external activating coil, and operating the external activating coil to inductively induce current flow in the activation coil with the capsule.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1A is a schematic illustration of an exemplary screening system in accordance with various aspects of the disclosure;

FIG. 1B is a schematic illustration of an exemplary external data-recording unit of the system of FIG. 1A in accordance with various aspects of the disclosure;

FIG. 2 is an illustration of an exemplary fail-safe imaging capsule according to various aspects of the disclosure;

FIG. 3 is an illustration of a capsule with an external activating coil according to various aspects of the disclosure;

FIG. 4 is an illustration of a capsule being activated by an external activating coil according to various aspects of the disclosure; and

FIG. 5 is a schematic illustration of an electronic activating circuit in the capsule according to various aspects of the disclosure.

### DETAILED DESCRIPTION

Reference will now be made in detail to specific embodiments or features, examples of which are illustrated in the accompanying drawings. Generally, corresponding or similar reference numbers will be used, when possible, throughout the drawings to refer to the same or corresponding parts.

Reference is made to FIG. 1A, which is a schematic illustration of a screening system 141, in accordance with various aspects of the disclosure. The system 141 typically comprises an ingestible capsule 150 and an external data-recording unit 152. For some applications, the data-recording unit 152 (FIG. 1B) may be worn on the waist of a subject 154 (as shown in FIG. 1A) or elsewhere on the subject's body, such as the wrist (configuration not shown), etc. Alternatively, for some applications, the capsule 150 may comprise an internal data-recording unit, and the external data-recording unit 152 may not be provided. In these applications, the data recorded by the capsule 150 is retrieved after the capsule has been expelled from the body.

During a typical screening procedure using system 141, an oral contrast agent 170 is administered to subject 154. Contrast agent 170 is typically adapted to pass through a gastrointestinal (GI) tract 172 and be expelled with the feces, substantially without being absorbed into the blood stream. The contrast agent material may be similar to compounds used routinely for the study of the GI with X-rays, such as Barium sulfate liquid concentrate, iodine-based compounds, or other such materials. For some applications, additional appropriate contrast agents include Tantalum, Gadolinium, Thorium, Bismuth, and compounds of these materials. After the contrast agent is administered (e.g., several hours after the contrast agent is administered), subject 154 swallows capsule 150.

Capsule 150 travels through GI tract 172, emitting gamma and/or X-ray radiation. Beginning at a certain point in time, capsule 150 records the Compton scattered gamma and/or X-ray photons that strike one or more radiation detectors 162 (FIG. 2). The count rate information received from each of the radiation detectors is typically stored together with a time stamp for that measurement. Within a time period typically of less than one second (e.g., several tens to several hundred milliseconds), it is assumed that the capsule and the surrounding colon wall and the contrast agent are in quasi-steady state. Taking small enough time intervals and integrating the counts over the small intervals allows for this quasi-steady-state assumption. The data may be stored in the capsule and sent by the capsule to the external recording unit from time to time, or after data-gathering has been completed.

Reference is now made to FIG. 1B, which is a schematic illustration of the external data-recording unit 152, in accordance with an exemplary embodiment of the present disclosure. The data-recording unit 152 may comprise a receiver/memory unit 155, a support electronics/battery unit 156, an antenna 157, and/or user controls 158. In some aspects, the unit 152 may also include a strap 159, such as a belt or wrist/arm strap, for coupling the unit to the subject 154.

Reference is now made to FIG. 2, which is a schematic illustration of a perspective view of an exemplary failsafe imaging capsule 100, according to various aspects of the disclosure. Various embodiments of failsafe imaging capsules are illustrated and described in U.S. patent application Ser. No. 13/821,999 (U.S. Patent Application Publication No. 2013/0172740), filed Mar. 11, 2013, U.S. patent application Ser. No. 13/895,345 (U.S. Patent Application Publication No. 2014/0037069), filed May 15, 2013, and U.S. Provisional Patent Application No. 61/___,___, filed on ______, entitled “LINEAR FAIL SAFE RADIATION CONCEALMENT MECHANISM” by Yoav Kimchy, all of which are incorporated herein by reference.

In an exemplary embodiment of the invention, a patient first swallows a contrast agent which mixes with the content of their gastrointestinal tract to increase the accuracy of the measurements. Then the patient swallows imaging capsule 100 to examine the gastrointestinal tract as imaging capsule 100 proceeds through the gastrointestinal tract. In an exemplary embodiment of the invention, imaging capsule 100 is designed to automatically block radiation from being emitted from it until receiving instructions to release radiation and image its surroundings. In an exemplary embodiment of the invention, no power is required to prevent blocking emission of radiation. Thus, if imaging capsule 100 lacks power the radiation will be blocked.

In an exemplary embodiment of the invention, imaging capsule 100 includes an encasement 105 for holding and protecting the elements of the device from acids and other liquids or gases along its path of motion. Optionally, the encasement should be able to withstand external pressures for at least 50-100 hours to allow for imaging capsule 100 to traverse the gastrointestinal tract and exit while still intact. Inside encasement 105, imaging device 100 includes a power source 180 (e.g. one or more batteries), a motor 185, a radiation source 110, a detector 195, a transceiver 135, and a controller 199. The controller 199 may be programmed with instructions for operating all the capsule functions, including operation of the transceiver 135. In an exemplary embodiment of the invention, radiation source 110 is located on a rotatable disk 145 and provides radiation that is blocked by a filling material 130 that forms the disk (e.g. made of lead or tungsten or other dense materials). Optionally, the radiation is only free to travel in a few specific directions through collimators 120.

In an exemplary embodiment of the invention, power source 180 provides power to motor 185, motor 185 is configured to rotate disk 145 around a rotation axis 125 with radiation source 110 and collimators 120 mounted on disk 145. In some embodiments, the collimator may be non-rotating, and the motor may be configured to move an arrangement linearly to block/unblock beams emitted from the radiation source and/or non-rotating collimator.

Optionally, one or more directed radiation beams are emitted from collimators 120 controllably scanning the surroundings through imaging capsule 100. Optionally, detector 195 detects backscattered particles resulting from the directed radiation beam. In an exemplary embodiment of the invention, detector 195 counts the detected particles and provides the information to transceiver 135 for transmission to an external device (e.g. a computer) for processing and optionally constructing a visual representation of the information. In some embodiments of the invention, transceiver 135 uses radio frequency (RF) transmissions to receive instructions from an external device and to provide information to the external device. Optionally, the external device may instruct imaging capsule 100 to start scanning, to stop scanning, and/or to scan in a specific motion pattern or at specific times.

It should be appreciated that the radiation source 110 may be adapted to emit gamma rays, X-rays, and/or beta electrons (i.e., radiation having energy of at least 10 keV). For some applications, the radiation source 110 may comprise a radioisotope or a miniature radiation generator. In some aspects of the disclosure, radiation source 110 may comprise a miniature X-ray generator, such as those described in one or more of the following references: U.S. Pat. Nos. 6,134,300 and 6,353,658 to Trebes et al.; Haga, A. et al., “A miniature x-ray tube,” Applied Physics Letters 84(12):2208-2210 (2004); and Gutman, G. et al., “A novel needle-based miniature x-ray generating system,” Phys Med Biol 49:4677-4688 (2004). Such a miniature X-ray generator or X-ray tube may be used for radiation source 110 instead of a radioisotope to illuminate the colon contents with X-ray photons. Turning such a generator on and off as needed typically reduces exposure of the subject to radiation. In addition, the energy range can be better controlled and the flux may be higher for the on periods without increasing subject total exposure. It should be appreciated that the capsule 100 may include more than one radiation source 110. According to various aspects, the capsule 100 may comprise one or more gamma and/or X-ray radiation sources and/or sources of beta electrons, such as T1201, Xe133, Hg197, Yb169, Ga67, Tc99,Tc99m, In111, I131 or Pd100.

Referring now to FIG. 3, according to various aspects, a capsule activation system 302 includes the capsule 100 and an external activating coil 3. The capsule 100 may contain an activation coil 2 within the capsule 100. For purposed of clarity, the capsule 100 is shown in FIG. 3 with its other components removed. The external activating coil 3 may be electrically coupled with an alternating current source 4 via a switch 5. The switch 5 can be operable to provide electric current from the alternating current source 4 to the external activating coil 3 when desired. The external activating coil 3 is an open coil configured to receive the capsule 100.

FIG. 4 illustrates activation of the activation coil 2 within the capsule 100. As shown, the capsule 100 is placed in the open external activating coil 3. The switch 5 is closed such that current is permitted to flow from the alternating current source 4 through the external coil 3. The flow of current through the external coil 3 inductively induces current flow through the activation coil 2 within the capsule 100.

Referring now to FIG. 5, the current induced inside the capsule 100 via the external activating coil 3 is rectified and drives a load switch 6, such as, for example, switch TPS22913 from Texas Instruments. The load switch 6, in turn, drives current to the capsule controller 199. The controller 199 includes an embedded switch which, in turn, latches the port connected to the load switch 6 to keep the load switch 6 in on state even after the capsule 100 is removed from the external activating coil 3.

Once the load switch 6 is on and the controller 199 is on, the embedded switch in the capsule controller 199 activates all the capsule functions, including operation of the RF transceiver 135, which can then be used to send and receive messages and instructions to and from the capsule 100.

From the foregoing, it will be appreciated that, although specific embodiments have been described herein for purposes of illustration, various modifications or variations may be made without deviating from the spirit or scope of inventive features claimed herein. Other embodiments will be apparent to those skilled in the art from consideration of the specification and figures and practice of the arrangements disclosed herein. It is intended that the specification and disclosed examples be considered as exemplary only, with a true inventive scope and spirit being indicated by the following claims and their equivalents.

What is claimed is:

1. An imaging capsule activating system, comprising:
an imaging capsule containing
a radiation source,
an arrangement with the radiation source mounted thereon, wherein the arrangement is configured to selectively allow emission of radiation from the radiation source,
a blocking arrangement configured to selectively block emission of radian from the radiation source, wherein in a rest position the emission of radiation from the arrangement is blocked,
a movement arrangement configured to move the blocking arrangement relative to the arrangement,
a controller configured to operate the radiation source and the blocking arrangement, and
an activation coil electrically coupled with the controller, the activation coil having an initial configuration without current flow; and
an external activating coil, the external activating coil being configured to receive the imaging capsule, the external activating coil being operable to inductively induce current flow in the activation coil within the capsule.

2. A method of activating an imaging capsule, comprising:
providing an imaging capsule containing
a radiation source,
an arrangement with the radiation source mounted thereon, wherein the arrangement is configured to selectively allow emission of radiation from the radiation source,
a blocking arrangement configured to selectively block emission of radian from the radiation source, wherein in a rest position the emission of radiation from the arrangement is blocked,
a movement arrangement configured to move the blocking arrangement relative to the arrangement,
a controller configured to operate the radiation source and the blocking arrangement, and
an activation coil electrically coupled with the controller, the activation coil having an initial configuration without current flow; and
disposing the imaging capsule in an open external activating coil; and
operating the external activating coil to inductively induce current flow in the activation coil with the capsule.

3. An imaging capsule activating system, comprising:
an imaging capsule containing
a radiation source,
a rotatable disk with the radiation source mounted on the disk and wherein the rotatable disk is configured to allow emission of radiation from the radiation source substantially only from a few locations on the circumference of the disk,
an outer ring surrounding the circumference of the disk and configured to rotate relative to the disk, the outer ring including areas that block radiation and areas that are transparent to the emission of radiation, wherein in a rest position the outer ring is situated relative to the rotatable disk such that the areas that block radiation are blocking the emission of radiation from the few locations on the circumference of the disk that allow the emission of radiation,
a rotation arrangement configured to rotate the rotatable disk relative to the outer ring,
a controller configured to operate the radiation source and the rotation arrangement, and
an activation coil electrically coupled with the controller, the activation coil having an initial configuration without current flow; and
an external activating coil, the external activating coil being configured to receive the imaging capsule, the external activating coil being operable to inductively induce current flow in the activation coil within the capsule.
